(19) United States
(12) Patent Application Publication
(10) Pub. No.: US 2012/0203125 A1
(43) Pub. Date: Aug. 9, 2012

(54) INHALATION MASK FOR ANIMALS

(75) Inventor: Declan Moran, Claregalway (IE)

(73) Assignee: NORTEV LIMITED, Claregalway, Co. Galway (IE)

(21) Appl. No.: 13/497,225

(22) PCT Filed: Sep. 17, 2010
(86) PCT No.: PCT/EP2010/063747

§ 371 Date: Apr. 20, 2012

(30) Foreign Application Priority Data
Sep. 21, 2009 (EP) 09170868.5

(51) Int. Cl. A61D 7/00; A62B 23/02; A62B 9/02; A61B 5/00; A61B 5/08; A62B 18/06; A61B 5/087

(52) U.S. Cl. 600/532; 128/206.24; 128/203.29; 128/206.12; 128/205.25; 600/538; 600/300

(57) ABSTRACT

An inhalation mask (1) for fitting to a snout of an equine animal or an animal having an equine shaped snout is provided and comprise: a unitary mask body (6) defining a housing with an open end (11) which is adapted to overfit and accommodate the snout of the animal. The open end (11) of the housing being an integral part of the housing and forming a deformable seal about the open end (11) for sealing about the snout of the animal, wherein the open end (11) of the housing comprises a deformable lip (10) which forms the deformable seal, the lip (10) being formed by a continuation of a side wall (7) forming the mask body (6), which when the mask (1) is fitted, turns back inward upon itself, into the open end (11) of the mask (1), so that the lip (10) engages with the animal's snout to support the mask body (6) on the snout.

### FIELD OF THE INVENTION

The invention relates to a drug delivery system, which may be used in veterinary applications. In particularly, the invention relates to a drug delivery system of the aerosol type, which are effective for treating equine or other livestock conditions, such as respiratory conditions, through targeted aerosol drug delivery to the lungs of the animal. One aspect of the invention is an inhalation mask suitable for use in such systems.

### DESCRIPTION OF RELATED ART

Animal livestock, including horses and cattle, can be susceptible to conditions affecting the respiratory tract. These animals are prone to such conditions as asthma, viral influenza, equine herpes and/or bacterial or parasitic infections of the respiratory tract. Any of these conditions are serious and they can adversely affect the animal resulting in poor performance of the animal. Athletic performance in the cases of recreational or performance sports horses, may be particularly affected. More serious cases can result in chronic pulmonary diseases, such as recurrent airway obstruction and/or pulmonary haemorrhage etc.

Conventional veterinary medical treatments include administration of anti-inflammatory steroids, antibiotics, antimicrobials, and/or bronchodilators and the like. Several disadvantages are associated with oral or injection methods of administration of such medicaments including systemic toxicity, precision associated with the amount of dose required for a particular respiratory treatment. An effective inhalation therapy can help avert financial losses related to valuable animals such as those which can occur for horse owners.

Aerosol drug delivery techniques are well known in the art and offers advantages over oral or injection techniques including superior and economical delivery efficiency, facilitation of use of lower dosages, specifically focused drug targeting resulting in reduced systemic side effects and system toxicity issues leading to reduced drug clearance times.

U.S. Pat. No. 5,429,599 teaches a syringe-based device for vaccinating an animal. The device has a nasal tip-cap seal which is designed to shield the needle as the syringe is inserted into the nostril of the animal, wherein the vaccine can be aspirated into the animal's nostril.

Canadian Patent Publication No. 2,043,188 describes an applicator for intranasal administration of vaccines to horses. The device comprises a long hollow tube for insertion into the nostril to reach the tonsilor tissue, and a blocking device that sits against the nostril. Vaccine is released in the form of small droplets on the tonsilor tissue of the horse.

Delivery using such devices may be difficult to achieve due to normal movement of the animal's head during attempting delivery. Thus it can be difficult to assess if correct dosing has occurred in the event the animal has become distressed.

While it is possible to sedate the animal, such a solution is not amenable to periodic or ongoing treatment, as the performance and/or overall health of the animal may be adversely affected.

U.S. Pat. No. 3,915,165 describes an intratracheal injection system for injecting dry medicaments (in a gaseous suspension) into the trachea of the animal for prevention of conditions such as pneumonia. Needle means are included as part of the system, and it is necessary to insert the needle into the trachea through the tracheal tissue before the dry medicament can be administered. The skilled person will appreciate that a method requiring intubation can be difficult, painful and are not intrinsically suited to regular, convenient use.

Later systems, such as described in European Patent No. 0 444 905, require insertion of a nasal tube into the nostril of the animal. When positioned correctly a lumen of the nasal tube communicates with the lung of the animal. An atomised aerosol drug may then be dosed into the lumen of the nasal tube to effect drug delivery from a connected canister, capable of delivering a metered dose.

Disadvantageously, these methods are highly invasive and potentially uncomfortable for the animal and generally not well tolerated. Where possible, it is desirable to avoid treatment techniques in which an animal would need to be sedated.

Equestrian and livestock inhalation devices facilitate delivery of medications to the lungs of horses and other large animals and their development have ameliorated problems with prior art methods.

U.S. Pat. No. 4,002,167 teaches an inhalation device in the form of gas mask assembly for an animal. The mask assembly has a nose cap member for covering the nostrils and anterior portion of the animal's snout. An opening is provided therein for attaching a breathing tube. A flexible sleeve member is secured to the nose cap and a cinch fixes the sleeve member against the snout to seal the sleeve thereto, so that gas can be delivered without leakage.

U.S. Pat. No. 7,111,626 describes an inhalation apparatus of the generally mask type. The mask is mountable on the head of a horse and comprising a valveless combined inhalation and exhalation port which includes a filter system which is mounted thereon. The mask design is said to seal when positioned on the horse's head, thereby preventing escape of aerosol and permitting a reliable inhalation of medicament. The seal is provided by a sealing ring fitted to the mask.

U.S. Pat. No. 5,954,049 teaches a mask body that fits over the nostrils of and mouth of a horse. An aerosolization chamber is secured to the body and opens into the interior thereof, and is supplied with misted medications (such as from a metered dose inhaler), for inhalation by the animal. The mask and body is also provided with one or more exhalation ports, and a headstrap or band or the like, for securing the body to the animal's face overlying the nostrils. The specification teaches that while the mask is somewhat flexible, it needs sufficient rigidity to prevent collapse against the horse's nostrils upon inhaling. The mask covers the nostrils and mouth of a horse which is lightly sealed to the adjacent areas of the horse's head but which allows a certain amount of air to pass under the seal along the horse's mouth as the document teaches that this is comforting to the horse. The mask is held in place by a conventional-type harness which includes straps which run behind the ears of an animal.

U.S. Pat. No. 6,349,725 teaches an aesthesia mask for animals. The mask has a body cavity defined to conform to the shape of the animal's face. It has an interface close to where the nose of the animal would be positioned for connecting to a breathing circuit. The mask also comprises a flexible and elastic seal member on the opposing end of the body, which tends to oppose deformation and conforms to the facial anatomy when in position.

United States Patent Publication No. 2004/0250816 describes an inhalation therapy mask and device for animals comprising a two-part mask. Each part of the mask is designed to fit over one nostril of the animal. The two parts of the mask must then each be fitted to the animal to allow delivery of therapeutic materials through both nostrils simultaneously. Each part of the mask has an aerosol chamber and an adaptation chamber separated by a dividing wall with an opening for the aerosol, so that, during the inhalation phases the aerosol passes together with the respirator air out of the aerosol chamber into the adaptation chamber. The adaptation chamber is to adapt the mask part to the body surface at the animal's respiratory opening. The adaptation chamber has an inwardly folded sealing lip with a free end, which provides flexible sealing of the mask at the animal's respiratory opening. The inward fold is configured so as to form a sputum collection area so as to restrict contamination. Furthermore, the seal prevents escape of medication about the respiratory opening and minimises the area of the body surface exposed to the aerosol so that virtually no aerosol can settle on the animal's head and face area to reduce medication waste. Since the adaptation chamber is designed to fit over the respiratory opening only, the seal is not sufficient for the mask to be self-supporting so as to hold itself on the animals face. The mask is supported by use of a conventional halter. The mouth of the animal is not within the mask.

U.S. Pat. No. 4,546,768 describes an animal inhalation apparatus, which includes a mask that is directly fastened to a vaporizer. The bag like mask has a portion of its surface disposed in the mouth of a horse and is secured by a tightening belt so that the horse is prevented from opening its mouth and is forced to breath through its nostrils, which lie inside of the bag.

International Patent Publication No. WO 2007/068341 describes a mask for aerosol therapy for delivering medication to a patient having a bowllike formation and the mask being provided with an opening, which comprises a facial contact surface. The opening is limited to the mouth contour and does not extend as to cover the nose of the patient. The arrangement allows nebulised product to be inhaled with the mouth and exhaled with the nose. The device is adapted for human rather than animal use. The mouth cavity of the patient is said to act as a homogenising chamber. In use the mask must be held against the patient's face as it is not self supporting when fitted.

Aerosol generating means such as a nebuliser head or indeed a metered dose inhaler (MDI) are useful ways of preparing an aerosol for administrating a medication to the lungs of an animal. However, it should be pointed out that both nebulisers and MDIs typically have a preferred orientation of operation for optimum operation.

In summary, a proportion of the inhalation apparatuses that are currently available contain inserts that must be fed into the animal's nose. During the administration of the medicaments, the inlet pipe, as well as the horse's head, must stay in a fixed position. Animals generally resist the insertion of the device and it can cause restlessness making administration difficult.

Another issue that arises is that, in use, mask apparatuses of poor design, may generate irregular and/or loud noises. Poorly designed masks may result in noisy or inconsistent air circulation through the mask. Badly designed valves may produce strange noises, and/or nebuliser generators may vibrate or produce further noise. Typical mask type systems require a connection to mains power, require a hose/tube or cable connection between the animal interface and aerosol generator or power supply. They are not very portable and increase the risk of poor drug administration to the animal. Animals generally find such devices unpleasant and disturbing, and find it difficult to accustom themselves to the procedures. The animal may become anxious and defensive in their behaviour, making treatment even more difficult. For example, the animal may start breathing irregularly, resulting in the medicaments insufficiently or not at all reaching the lungs. A further issue that arises in certain apparatus is that when the animal exhales, very often the medicaments are exhaled also and can enter into the inhaled air of the person tending to the horse.

Thus, it is desired to provide an improved portable inhalation based drug delivery system for snouted animals, with particular focus on treating members of the equine species. Provision of an improved portable inhalation drug delivery system, which would be suitable for either liquid drug nebulisation or with metered dose inhalers (MDI), would be particularly advantageous. It is also desirable that an improved drug delivery system would be compact and portable and not require connection by tubes or cables to other apparatus such as compressors, ultrasonic nebulisers or wall sockets. It is further desirable still that such systems are self-supporting on an equine-shaped snout without need for further parts or components for holding the system in place.

Improved integrated animal drug delivery system that is capable of applying highly efficient and accurately dosed medication into the animal's respiratory system in a properly atomised spray are of significant value.

### SUMMARY OF THE INVENTION

According to the present invention, as set out in the appended claims, there is provided an inhalation mask (1) for fitting to a snout of an equine animal or an animal having an equine shaped snout and comprising:

a unitary mask body (6) defining a housing with an open end (11) which is adapted to overfit and accommodate the snout of the animal so that, when fitted to the animal, a nose, mouth and anterior portion of the animal's snout are entirely within the housing, the open end (11) of the housing being an integral part of the housing and forming a deformable seal about the open end (11) for sealing about the snout of the animal,

wherein the open end (11) of the housing comprises a deformable lip (10) which forms the deformable seal, the lip (10) being formed by a continuation of a side wall (7) forming the mask body (6), which when the mask (1) is fitted, turns back inward upon itself, into the open end (11) of the mask (1), so that the lip (10) engages with the animal's snout to support the mask body (6) on the snout.

Inhalation therapy generally requires use of a suitable breathing device, such as a mouth piece device, face mask or other means held in close proximity to the patient's airways. A human patient can be taught to use a breathing device in the correct manner, for example, instructions can be give to place the mouth around a mouth piece or maintain a facemask in position. For unconscious or incapacitated patients, face masks can be held by an assisting person or can be strapped around the nose and mouth, if needs be.

Inhalation therapy for animals is problematic as the animal cannot be taught how to use a breathing device or anatomically they cannot be expected to hold a breathing device in position. Many of the prior art devices require continuous human intervention to maintain the device in the place while one or more doses are administered. Self-supporting devices would be desirable and advantageous.

Thus in one aspect there is provided an inhalation mask (1) for fitting to a snout of an equine animal or an animal having an equine shaped snout and comprising:

a unitary mask body (6) defining a housing with an open end (11) which is adapted to overfit and accommodate the snout of the animal so that, when fitted to the animal, a nose, mouth and anterior portion of the animal's snout are entirely within the housing, the open end (11) of the housing being an integral part of the housing and forming a deformable seal about the open end (11) for sealing about the snout of the animal,

wherein the open end (11) of the housing comprises a deformable lip (10) which forms the deformable seal, the lip (10) being formed by a continuation of a side wall (7) forming the mask body (6), which when the mask (1) is fitted, turns back inward upon itself, into the open end (11) of the mask (1), so that the lip (10) engages with the animal's snout to support the mask body (6) on the snout such that the mask is substantially self-supported on the animal's snout by an interference fit between the deformable lip and the animal's snout.

In a related aspect there is provide an inhalation mask (1) for fitting to a snout of an equine animal or an animal having an equine shaped snout and comprising:

a unitary mask body (6) defining a housing with an open end (11) which is adapted to overfit and accommodate the snout of the animal so that, when fitted to the animal, a nose, mouth and anterior portion of the animal's snout are entirely within the housing, the open end (11) of the housing being an integral part of the housing and forming a deformable seal about the open end (11) for sealing about the snout of the animal,

wherein the open end (11) of the housing comprises a deformable lip (10) which forms the deformable seal, the lip (10) being formed by a continuation of a side wall (7) forming the mask body (6), which when the mask (1) is fitted, turns back inward upon itself, into the open end (11) of the mask (1), so that the lip (10) engages with the animal's snout to support the mask body (6) on the snout such that the mask covers substantially large portion of the snouted animal's face, wherein the mask grips the animal's face sufficiently to be self supported thereon.

The unitary mask of the invention provides a substantially airtight mask, which is shaped to fit securely over the nose and mouth of a snouted animal such as an equine or an animal having an equine shaped snout. The nature of the mask of the invention is such that the mask has a sealing lip which engages with the animal's snout such that when fitted the mask body grips the animal's snout to support the mask on the animal's snout. In other words, the lip engages with the animal's snout to such that the deformed lip sufficiently grips the snout to support the mask (1) when it is correctly positioned on the animal's snout. When fitted, the mask is substantially self-supporting on the animal's snout. By self-supporting it is meant that the fitted mask, remains by itself on the animal's snout, generally by way of an interference fit between the deformable sealing lip and the animal's snout/upper portions of the animal's face. The mask will remain in position without use of further securing means. The shape of the mask allows this type of fit.

The mask body is designed to cover entirely the mouth, the nostrils and anterior portion of the animal's snout. The inhalation mask of the invention desirably has a generally “racetrack” or “stadium” shaped when viewed from above. By racetrack or stadium shaped, it is meant that when viewed from above the mask has substantially parallel sides with ends shaped to mate to the profile of the snout of the animal. For example, the mask may have parallel sides, with ends which are curved. The shape is important since it facilitates the self-supporting aspect of the mask when it is fitted in position on the animal's snout. To assist further in the self supporting nature of the mask, the racetrack or stadium shape desirably tapers inward from it maximum size at the opening of the mask to the bottom of the mask where the animal's nostrils will be positioned. In other words, the mask tapers inward to complement the shape of the animal's snout. This can assist the mask to sit securely in a substantially self-supporting manner when fitted. The animal is then free to move about while being treated. This has a more natural and calming feel to the animal. Animals have an elongate head where the nose/mouth are at an anterior end, and the eyes and ears are closer to a posterior part of the head with a substantial distance between the anterior and posterior parts.

The inhalation mask of the invention is suitable for fitting to and for treating snouted animals. By snouted, it is meant to describe animals such as equine animals, or animals having an equine type snout. By equine type snout, it is mean, those animals having a projecting nose or nostrils, jaws, or anterior facial part of an animal's head. More particularly, the mask may be used with snouted animals having equine snouts such as equine family members, including horses and ponies. Animals having equine type snouts as defined herein include canines, such as dogs, buffalo or cattle and the like, camels, llamas and the like. When the mask is fitted to the snout of the animal, it is critical that the mask entirely covers the mouth, the nostrils and anterior portion of the animal's snout. It is desired that the mask covers substantially large portion of the snouted animal's face, so as to sure that the mask grips the animal's face sufficiently to be self supported thereon. In some masks, the mask body walls may extend up to just below the eyes of the animal. The mask must extend upwards onto the animal's face to, at a minimum, the area where a substantially good seal can be formed between the animal's face and the mask body. The skilled person will appreciate that the mask desirably should stop short on the animal's face, of areas where there are grooves or cheek hollows or other indented areas, which are depressed or sunken, or are shaped such that a sufficient seal between the deformable lip and the animal's face cannot be formed. Due to the relative expense of such medicinal treatments, (e.g., cost of medications, cost associated with operator training), it is likely that the inhalation mask will be most suitable for treating high value animals, such as thoroughbred cattle, camels and horses etc.

In a preferred embodiment, the mask body is of a unitary construction. Advantageously, the inhalation mask of the invention is a unitary mask which has a mask body that is formed from a single piece of material. Desirably, the mask fits to the animal in a nose-bag type shape or inverted funnel shapes, that is, accommodating the nostrils, nose and mouth but with room about the snout to have an atmosphere of material to be inhaled. Many existing masks generally have more than one part which may comprise at least firstly, a mask shell, and additionally a separate sealing part or section and/or further components. Such multipart devices require assembly before use and disassembly before cleaning and/or disinfection. These types of masks are inherently more difficult to clean and consequently pose a greater risk of contamination. Bacteria can lodge in crevices and joints between sections (e.g. where the shell and seals meet, mate or inter-engage). Furthermore, masks having more than one piece are more complex to fit and accordingly training and time may be require fit the apparatus correctly. For example, a seal part may have to be positioned initially before the entire mask body can be securely fitted. In a busy or unorganised stable environment, critical pieces may easily be lost or mislaid, rendering the device unusable. Accordingly, the one-piece design of the mask body of the present invention with integrated seal is an advantageous design that facilitates fitting the device on the animal in a relatively quick and straightforward manner overcoming problems with prior art masks which are typically constructed of many separate parts meaning they are difficult and time consuming to assemble and difficult to clean and service. The unitary, one-piece design allows easy cleaning and disinfection between uses. The mask of the invention is advantageous since it does not require an additional separate sealing part to be added to the mask before it can be fitted. A number of prior art mask require a rubber seal or the like to be fitted to a generally harder mask shell portion before a seal can be formed between the mask and the animal's face. In the mask of the invention an interior side of the lip forms the seal.

Suitably, the inhalation mask of the invention may be substantially nose-bag shaped, so as to entirely fit in a snug manner about the snout (nostrils, nose and mouth) of the animal to be treated. In a preferred embodiment the inhalation mask substantially (inverted) funnel shape, tapering inwards from the opening at the upper end of the mask to the narrower nose portion where, in use, the mouth of the animal will be positioned. The tapering shape is desirable since it is one simple shape that allows the mask to be positioned in a self-supporting matter when fitted, wherein the sealing deformable lip grips the animal's snout in manner to support the mask. Furthermore, a snug fit ensures that dead space within the animal inhalation mask interior is minimised. This will assist in avoiding carbon dioxide and exhaust/expiration waste build up. In a related embodiment, the mask may be used to prevent feeding or biting as the mouth of the animal is enclosed within the mask. For example, the mask of the invention may be put in place for such a purpose without using it for administering a therapeutic material. It can also be left in place between administration of doses of therapeutic materials.

Suitably, the inhalation masks of the invention may be provided in a number of sizes, depending on the anatomy/age of the animal to be treated. For example, in the case of an equine inhalation mask, mask sizes can be provided which are suitable for foals (aged from 0 to 6 months), fillies and colts (aged from 6 to 18 months), to adult horses.

In an alternative embodiment, there is provided an inhalation mask for fitting to a snout of an animal and comprising:

a mask body defining a housing with an open end which is adapted to overfit and accommodate the snout of an animal so that, when fitted to an animal, a nose and mouth of the animal are within the housing,

the open end of the housing comprises a deformable lip that forms the seal when the mask is positioned correctly on the animal's snout.

Suitably, the lip may be formed by a continuation of a wall forming the mask body and which turns back upon itself.

Advantageously, the open upper end of the housing may comprise a deformable lip which turns inwardly to form an inverted lip or rimmed edge. Typically, the deformable lip is formed by a continuation of a wall forming the mask body and which turns back upon itself and into the open end of the mask.

In preferred arrangements, the deformable lip of the housing turns inwards towards the interior of the inhalation mask. Suitably, the deformable lip or rimmed edge of the housing may be inwardly turned in a curved manner or in substantially angled manner to form an inner inward turned wall portion of the mask body. Desirably, this wall forms the seal. In one embodiment, the deformable lip or rimmed edge of the housing may be angled in a position substantially perpendicular to the mask body wall. In a preferred embodiment, the deformable lip or rimmed edge of the housing is inwardly turned in a curved manner such as to form an open inverted substantially U-shaped inwardly turned surface or lip. The inverted U-shape is desirable, since the U-shape formed allows sufficient deformation of the lipped or rimmed edge to occur such as to ensure a good seal between the lipped or rimmed portion of the mask body wall against an inserted snout. It should be appreciated that since the mask is fabricated from a flexible material, the lip can in practice, be present before or indeed after the mask has been fitted. While the present invention focuses primarily on masks having a deformable inwardly curved lip when the mask is in the rest or unfitted state, it falls within the inventive concept to have a mask where the inwardly turned lip or rim only forms as the mask is fitted onto the animal's snout. In other words the lip does not have to be formed into an inwardly curved lip when the mask is not in use, it is sufficient that the sealing lip can be arranged to be inwardly turned on being fitted on the animal's snout.

In one embodiment, there is provided an inhalation mask for fitting to a snout of an equine animal or an animal having an equine shaped snout and comprising:

a mask body defining a housing with an open end which is adapted to overfit and accommodate the snout of the animal so that, when fitted to the animal, a nose, mouth and anterior portion of the animal's snout are entirely within the housing, the open end of the housing being an integral part of the housing and forming a deformable seal about the open end for sealing about the snout of the animal,

wherein the open end of the housing comprises a deformable lip which forms a deformable seal, the lip being formed by a continuation of a side wall forming the mask body, which when the mask is fitted, turns back inward upon itself, into the open end of the mask to form an air trap between the inwardly turned wall surface of the deformable lip and the inner wall of the mask body side wall.

The skilled person will appreciate that in respect of the minimum length of the inwardly curved portion of the mask body wall necessary to form a sufficiently good seal, the minimum length is any length which is sufficient to allow an inward turn of the wall to be made to result in formation of the sealing lip or rimmed edge such that the inwardly turned portion forms a gap, that acts as an air trap, between the inwardly turned wall surface of the deformable lip and the inner wall of the mask body side wall. This space (for example, formed by the bottom of the U-shape) acts as an air trap when the animal exhales and back pressure of air trap in the air gap further seals the mask to the animal's snout in a desirable manner.

In a related aspect, there is provided a seal for an inhalation mask comprising a deformable lip, which forms a deformable seal being formed by a continuation of a mask sidewall, which when the mask is fitted, turns back inward upon itself into the mask, forming an air trap between the inwardly turned wall of the deformable lip and an inner wall of the mask body side wall. The skilled person will appreciate that the air trap acts to substantially seal the mask wall to the animal's snout. It is apparent that the air trap aspect of the seal of the invention may be applied to other masks. Prior art masks generally have a solid sealing ring or like which seals the mask wall to the animal's face. The advantage of a seal created by an inwardly turned deformable rim is to provide an air trap, which can utilise back pressure to create the seal.

The minimum length of inward curved wall required to form the air trap space will depend on the size of the mask and the animal's face to which the mask is to be fitted. The minimum length is that length necessary to create a sufficient seal between the inwardly turned mask wall and the animal's snout. This assists to substantially seal the deformable inwardly curved lip portion of the mask body wall against the animal's snout. The effect may be to catch sufficient backpressure, so as to give a better seal. This in turn ensures that substantially none of the aerosol escapes between the inwardly curved lip and the animal's snout. Preferably, at least 1.5 cm of the wall forming the mask body forms the inwardly turned deformable lip. More preferable still, at least 3.5 cm of the wall turns into the interior of the mask, most preferably yet, at least 4 cm of the wall turns into the interior of the mask to form the inwardly turned deformable lip, which is of sufficient length away from the where the inward turn commences to ensure deformation occurs such as to seal the mask against an inserted snout. The length measurement is measured from the point on the mask body wall, where the curved inward portion of the mask body wall starts to turn inwards—In other words from where the mask body wall starts to deviate from being substantially linear.

The seal design is advantageous, since it allows the deformable inwardly turned lip to positively engage with the upper portions of the animal's snout when the inhalation mask is placed in the optimum position to create a seal. When positioned correctly when the animal exhales, the deformable inner housing wall substantially seals the mask onto the animal's snout. The seal design is such that when the animal exhales any back pressure generated has the effect of improving the seal against the animal's snout as the internal pressure builds up in the U-section of the seal and forces the sealing lip around the snout of the animal. Advantageously, the corresponding/complimentary shapes of the animal's head and the mask body assists in making a soft but effective seal. In use, when the animal breathes out, backpressure created improves the seal. A sufficient seal is desirable, since this ensures that substantially none of the aerosol, when delivered, is lost between the sealed lip and the animal's face.

Advantageously, the mask body of the inhalation mask of the invention is constructed of a material having sufficient flexibility to perceptibly rise and fall with respiration of an animal. Desirably, flexible material is such to ensure a lightweight mask housing that is rigid enough to hold and maintain its overall moulded shape while having sufficient flexibility to pulsate or slightly expand and contract with the animal's breathing cycle. Mask body pulsation or a perceptible rise and fall effect is an indicator of the existence of a good seal between the inwardly inverted sealing lipped or rimmed edge of the inhalation mask and the animal's snout. The mask body varies in wall thickness to achieve both rigidity and flexibility depending on the functional area of the mask body. Desirably, a flexible material also achieves a one-piece mask body with integrated flexible seal. Preferably, the inhalation mask body and/or housing may be constructed from a flexible thermoplastic elastomer (TPE) type material. Alternatively, the mask body may be constructed from a soft/flexible PVC type material. Inhalation masks made from these types of material are desirable since they ensure the mask not only has the required degree of flexibility, but that it is water resistant, unbreakable and shock proof. These materials of construction are relatively “clean” materials, as they are not formed of sticky polymers and accordingly do not tend to pick up dirt and dust (contrast with materials such as silicone). Furthermore, unlike materials of construction used in many prior art masks, e.g., made of rigid thermoplastic materials (such as polycarbonate), these materials are not prone to scratching or shattering if impacted such as dropped or hit. Desirably, the mask of the invention has a soft feel and the animal may find that it has a more natural texture and feel than harder mask housings, particular those made of more than one piece. In a particularly preferred embodiment, the animal inhalation mask of the invention is made from a thermoplastic elastomer type material such as a flexible polyurethane material. In an equally preferred embodiment, the animal inhalation mask of the invention is made from a soft PVC type material. Preferably, the flexibility of the material of construction of the mask housing will have a desirable Shore hardness. Hardness may be defined as a material's resistance to permanent indentation as measured by durometer. Desirably, the material of construction will have a Shore A hardness of between 65 to 95, for example 75 to 93, for example 80 to 90. This will provide the mask housing with the degree of rigidity and flexibility required to provide the advantageous features stated herein.

Desirably, the animal inhalation mask of the invention comprises at least one inlet for allowing air into the mask during inhalation by the animal and at least one outlet for allowing venting or expiration during exhalation by the animal. The at least one inlet and at least one outlet provided in the housing body receive and exhaust gas and/or aerosol respectively during use.

In a preferred embodiment, at least one inlet of the inhalation mask of the invention is valved to automatically open upon inhalation and automatically close upon exhalation.

In another embodiment, the inlet valve is mounted in the mask inlet. In a preferred embodiment, the inlet valve may be adapted to fit into an inlet insert, which may be specifically designed for mounting in the housing inlet of the mask housing.

In a preferred embodiment of the invention, the valved inlet is a one way valve which operates to only allow air/aerosol or air and aerosol mixtures into the mask body.

In a preferred embodiment, at least one outlet of the inhalation mask of the invention is valved to automatically close upon inhalation and automatically open upon exhalation.

In another embodiment, the outlet valve, is mountable in the mask outlet. In a preferred embodiment, the outlet valve may be adapted to fit into an outlet insert, which may be specifically designed for mounting in the housing exhaust outlet of the mask housing.

In a preferred embodiment of the invention, the valved outlet is a valve which operates to only allow exhaust/aerosol or air and aerosol mixtures out of the mask body.

In a preferred embodiment, the inlet of the animal inhalation mask of the invention comprises at least one of additional components selected from the group comprising an inlet valve, an aerosol chamber, a dust/bacterial/viral filter, a gas inlet adaptor and an inlet gas measuring device such as a flow sensor.

Additionally, the outlet of the animal inhalation device of the invention comprises at least one of additional components selected from the group comprising an outlet valve, a particle filter such as a dust/bacterial/viral filter, a flow sensor, an exhaust aerosol filter and an exhaust gas-measuring device.

Suitably, these optional components provide specific advantageous if used, e.g., an aerosol filter is useful when potentially hazardous substances are administered and exhaled in the vicinity of the handler. Similarly, if a flow sensor component or exhaust measuring device is included, this will facilitate measuring gases/breath of the animal which may be useful in assessing fitness, lung capacity etc.

Preferably, the inhalation mask housing may comprise a bypass port system, optionally adjustable, for allowing unhindered intake and exhalation of air from the mask when necessary. The bypass port system allows bypass of the valved inlet and outlets and provides a direct opening in the mask. The bypass port system, when activated, quickly overrides the inlet/outlets to allow air/exhaust into and out of the mask by bypassing the valve arrangements. A bypass port system and in particular an adjustable one, is of benefit in cases of nervous or excitable animals, since activation of same, will allow the animal to breath more freely and normally (without any restriction which may be experienced while breathing through the valved inlet and valved outlet which may be connected to other devices), thereby alleviating stress. Making it adjustable further allows the tuning of the air allowed through the bypass system. Activation of the bypass port system is useful when introducing the mask to the animal for the first time. Preferably, such a bypass port and its adjustable valve system are preferably located towards the bottom of the mask proximate to the horse's mouth to facilitate a more relaxed breathing environment, if so needed.

Desirably, the bypass port comprises at least one slot or opening, positioned in the mask body to provide direct access to the exterior environment. Suitably, the bypass port slot or opening is positioned in the mask body toward the bottom of the mask in the vicinity of the animal's mouth and nostrils. In a preferred embodiment, the bypass port slot or opening comprises two opposing semi circular shaped slots or channels.

The bypass port system may be provided with a valve means. The valve means can be utilised to open or close the bypass port or when adjustable to open or close to varying desirable degrees, as required.

In a related aspect of the invention, there is provided a bypass port system comprising:

a rotatable insert body mountable in an inhalation mask body for rotation thereon; and

a valve means comprising at least one flange extending outwards from the body and adapted to reversibly obstruct at least one opening in the inhalation mask body

such that rotation of the insert body moves the at least one flange from a closed position in which the inhalation mask body opening is substantially closed to a bypass position in which the inhalation mask body opening is substantially open.

In a preferred embodiment, the rotatable bypass port insert body is removeably mounted within the bypass port. The at least one flange may extend outwardly from the rotatable insert body. Desirably, at least one flange is substantially complementary to bypass port slots or openings positioned on the inhalation mask body.

When the rotatable bypass port valve insert body is rotated, the at least one flange also rotates and can be rotated into position to engage with the bypass slots to create a seal that closes off the bypass slots or openings. The system comprising mask slots and insert body flanges together form an adjustable bypass port valve can be activated and deactivated by rotating the insert to engage or disengage the flanges from covering the slots. The rotatable adjustable bypass port valve system may be activated/deactivated by a convenient and easy to operate twist function. Due to the rotation action, the bypass valve can be fully opened or fully closed or may be set to any intermediate position as necessary.

In a particularly preferred embodiment, the mask outlet and bypass port slots or openings can be the same. Desirably, the mask outlet and bypass outlet can be arranged to complement each other in operation. Thus accordingly, incorporating both features together minimises the number of exit valves, for example, to just one and makes the mask easier to clean between uses. In this embodiment, the bypass port system comprises a rotatable bypass port valve insert, which is insertable into the inhalation mask outlet.

Thus, in one embodiment, the rotatable bypass port valve insert comprises:

a rotatable insert body for insertion into an opening on an inhalation mask body having at least one flange extending outwards from the body, the flange being adapted to reversibly obstruct at least one further opening in the inhalation mask body; and

a frame located within the insert body adapted for mounting a inhalation mask outlet valve thereon;

such that when the insert body is positioned in the opening, the insert body and valve are rotatable therein to move the at least one flange from a closed position in which the inhalation mask body opening is substantially closed to a bypass position in which the inhalation mask body opening is substantially open.

In another embodiment, the animal inhalation mask further comprises a mask securing means for securing the mask to the animal's snout. Suitably, such means comprises a headstrap, which preferably is adjustable. The headstrap may be connected on opposing sides of the mask through fastening means such as hooks, eyelets, hook and loop fasteners such as those sold under the trade mark Velcro, snap fit connectors or the like as will be known to the person skilled in the art. Such means may be located on the headstrap or the mask securing means. It is preferable that such fastening means is safe, non-irritating to the animals skin and is of a quick release nature in the event the mask has to be removed in urgency. The skilled person will appreciate that depending on the type of fastening means used, the mask housing will comprise the opposing cooperating connector. The headstrap may have a further adjustment means such as a belt and buckle arrangement remote from the mask.

In a preferred embodiment, the inhalation mask further comprises an aerosol chamber in which aerosolized material can be present for mixing with air prior to being inhaled.

In a particularly preferred embodiment, the aerosol chamber is external to the mask and is in communication with the housing of the mask. Desirably, the aerosol chamber may be removable from the mask or may be permanently attached thereto. However, it is preferred that the aerosol chamber is removeably mounted on the mask housing since this facilitates mask and aerosol chamber cleaning and allows the mask to be used for other applications, such as oxygen or other gas delivery, flow sensing applications etc.

Advantageously, the aerosol chamber is moveable relative to the mask so that the relative position of the chamber to the mask can be changed while the chamber remains in communication with the housing of the mask.

Desirably, the aerosol chamber is rotatably moveable relative to the mask housing. Accordingly, the invention provides an aerosol chamber for an animal inhalation mask comprising:

a chamber portion having an outlet for passage of aerosol into the mask and an air inlet adapted for intake of a substance from a delivery device,

wherein the chamber portion is rotatable with respect to the mask, so that the position of the inlet relative to the mask is rotatably adjustable.

Desirably, rotation allows the height of the inlet relative to the mask to be adjusted. Rotation may be about the chamber outlet. In particular, it is desirable that the chamber portion has an approximate L-shaped configuration with the inlet opening and outlet openings at each end of that L-shape. This allows for substantial relative movement of the inlet relative to the mask.

The aerosol chamber of the invention can be connected to the mask when required for use and can be rotated to different positions depending on the mode of use and aerosol drug delivery method required. For example, if a nebuliser is to be used to generate the mist or aerosol, the aerosol chamber can be orientated to be substantially vertically disposed relative to the mask. If a metered dose inhaler is to be used as the aerosol drug delivery device, then the aerosol chamber can be rotated substantially 90 degrees to the vertical to be disposed in a substantially horizontal position (in the fitted position of the mask). This will allow an MDI to be used to generate the aerosol, the MDI being operable in its optimum vertical position. An MDI will not function correctly when the aerosol chamber is in the vertical position, as the MDI would have to be held horizontally to engage with the aerosol chamber. The arrangement provides improved accessibility and optimum position for using a MDI correctly to ensure correct dosage is administered.

Rotational movement of the aerosol chamber on the mask is desirable since the arrangement eliminates the necessity for extra accessories to accommodate either the nebuliser or the MDI, thus providing an integrated solution without needing additional plug on parts for use with the nebuliser head or an MDI. It is preferred that the aerosol chamber rotates so that the animal can be approached from its left hand side. The skilled person will appreciate that generally it is usual to approach a horse from its left hand side and so rotation in this direction is sensible. However, the chamber can rotate both sides if so desired.

In a preferred embodiment, the aerosol holding chamber is made from a material which is sufficiently transparent to allow chamber loading and emptying to be observed through the walls thereof. Transparent or clear material is thus useful.

In a preferred embodiment the aerosol chamber is substantially L-shaped.

In another embodiment, the aerosol chamber comprises an optional aerosol outlet valve. Such an aerosol outlet valve protects and contains the generated mist (aerosol) while making it readily available for inhalation through the mask during the inspiratory cycle of the animal when the chamber is connected thereon. Suitably, the aerosol outlet valve may be a one-way valve.

In one embodiment, the aerosol outlet valve can be provided separately for use with a mask inlet or it may be an integrated part of the aerosol holding chamber. However, the latter embodiment is preferred, since it means the aerosol can be prepared and stored in the aerosol holding chamber remote from the animal and can simply be fitted to the mask inlet when required.

In another preferred embodiment, the aerosol chamber comprises an air inlet valve. The aerosol holding chamber preferably has an air inlet valve integrated into the chamber assembly to prevent leakage of the aerosol to the environment. Suitably, the air inlet valve may be a one-way valve. This is a preferred feature since it minimises the risk of passive inhalation by the caregiver and other nearby animals etc. Thus, the valved aerosol chamber of the invention protects and contains the generated mist (aerosol) while making it readily available for inhalation through an aerosol inlet valve during the inspiratory cycle of the animal.

In a particularly preferred embodiment, at least one inlet for allowing air into the mask during inhalation by the animal is provided on the aerosol chamber. In such an embodiment, the at least one inlet can be provided in a vertical wall of the chamber or if an aerosol chamber cap is provided, then the at least one inlet may be provide on the top of the chamber cap (horizontal position). It is preferred that the aerosol inlet is located in the top of the chamber cap as the airflow into the chamber is more efficient when the inlet is in a substantially horizontal position.

The air and aerosol valves open when the animal inhales allowing air and the generated aerosol into the animal's airways and lungs.

In a preferred embodiment, the aerosol holding chamber of the invention further comprises a nebuliser head which desirably further contains a medication cup for a liquid substance such as a drug, typically a fixed volume of liquid drug. Alternatively, the nebuliser may be connected to a continuous supply such as a feedbag to provide a continuous feed to the nebuliser. This may be accomplished by providing a special nebuliser cap that accommodates connection to a supply, such as, an IV drip bag or the like. Desirably, the aerosol generator may be a nebuliser such as a spray nebuliser or an ultrasonic nebuliser or the like.

In an alternative embodiment, there is provided an inhalation mask for fitting to a snout of an animal and comprising:

a mask body defining a housing with an open end which is adapted to overfit and accommodate the snout of an animal so that, when fitted, a nose and mouth of the animal are within the housing; and

an aerosol chamber in which aerosolized material can be present for mixing with air prior to being inhaled;

wherein the aerosol chamber is moveable relative to the mask so that the relative position of the chamber to the mask can be changed while the chamber remains in communication with the housing of the mask.

In a preferred embodiment of this aspect, the mask body comprises a deformable seal about the open end for sealing about the snout of the animal.

In a further preferred embodiment, the mask further comprises a bypass port system.

In a further preferred embodiment, the mask further comprises a rotatable aerosol holding chamber.

In a preferred arrangement, the mask body is provided with an attaching means for attaching an electronic nebuliser controller or the like to the mask. Suitably, the attaching means is a pocket, substantially dimensioned to accommodate the controller device securely, although the skilled person will appreciate that any attachment means capable of holding a controller in position on the mask may be suitably used. Mask/controller attachment means is desirable since it means that if a battery operated controller device is used, then the mask does not have to be attached to power sockets or generators or the like. This is advantageous, since the mask is portable and the animal can be allowed to move around as it pleases while the medication is being administered. Typical mask type systems require a connection to mains power, require a hose/tube or cable connection between the animal interface and aerosol generator or power supply. They are not very portable and increase the risk of poor drug administration to the animal.

With reference to the various specific embodiments described herein, it is important to point out that particular advantages arise from combining one or more features of any of the embodiments of the above invention. Combinations of one or more of the feature is possible and provides an optimised animal inhalation mask. Any particular combination of the features as set out in the claims and in the description is possible and specific combinations will provide particular advantages. In particular, it will be appreciated that an animal inhalation mask of the invention can incorporate any combination of the features described. While the present inventors have made many independent improvements, it will be appreciated that each of the improvements can be used in combination with any of the others, particular those features mentioned independently. It is particularly advantageous for example, to combine the deformable inwardly curved sealing lip feature with the rotatable aerosol holding chamber feature and further advantageous to combine either of these features alone or in combination with the bypass port system and in further combination or sub-combination with unitary mask body feature or any other sub-combination or permutation of the independent features.

### BRIEF DESCRIPTION OF THE DRAWINGS

The invention will be more clearly understood from the following description of an embodiment thereof, given by way of example only, with reference to the accompanying drawings, in which:—

FIG. 1 shows an animal inhalation mask of the present invention in position on a horse's snout;

FIGS. 2A to 2C show top perspective and a front view of the flexible mask body and housing of the invention in various orientations;

FIGS. 3A and 3B show side and front section view along section line A-A of the flexible mask body and housing of the invention respectively;

FIG. 4 shows a top left perspective view of the inhalation mask having a rotatable bypass port valve-insert installed in position in the mask housing bypass port;

FIGS. 5A and 5B show top perspective views of the inhalation mask in an upright and inverted position respectively. The mask has an aerosol chamber installed in position on the mask housing body and a rotatable bypass port valve-insert installed in position in the combined bypass port/outlet of the mask;

FIG. 6A shows a side section view of the mask assembly along section line B-B as shown in FIG. 6B, showing through section of the aerosol chamber; FIG. 6B shows a front section view though mask assembly along Section line B-B of FIG. 6A, showing aerosol chamber cap an air inlet valve;

FIG. 7A shows a side section view of the mask assembly along line B-B of FIG. 7B, showing through section of the aerosol chamber with air inlet valve and air outlet valve in open position; FIG. 7B shows a front section view though mask assembly along Section line B-B of FIG. 7A, with air inlet valve in open position;

FIGS. 8A and 8B show section views of the mask assembly along line B-B, showing optional air/aerosol inlet valve in its closed position between aerosol chamber and mask body;

FIG. 9 shows a front perspective view of the mask assembly with the aerosol chamber rotated to the left side position for delivery of aerosol from a MDI;

FIGS. 10A and 10B show a left side elevation view and a front left perspective view of the inhalation mask with installed rotatable bypass port valve-insert in position in the mask housing bypass port and aerosol housing chamber in the vertical position respectively. Alternative positions for the aerosol chamber inlets (on a side wall of the aerosol chamber housing) are shown in this view; a blown up view of the one way inlet valve in the open and closed position is also shown;

FIG. 11 shows a front perspective view of the mask in side orientation illustrating the bypass port, bypass slots in the mask housing and the rotatable bypass port valve-insert installed and in the open position;

FIG. 12 shows a top left perspective view of the mask having the optional gas (oxygen) port adaptor inserted into the mask inlet; gas hose to the gas supply is also shown;

FIG. 13 shows a top left perspective view of the mask with a nebuliser head and nebuliser controller ready to be installed into position;

FIG. 14 shows a top left perspective view of the mask showing the optional plug in filter onto the bypass port rotatable valve-insert for capturing exhaled aerosols;

FIGS. 15A and 15B show perspective views of the mask having a nebuliser head installed into the nebuliser port on the aerosol chamber and controller for the nebuliser is installed into the pocket on the inhalation mask;

FIG. 16 shows an exploded view of the mask assembly;

FIG. 17A shows a cross sectional view of a fitted mask along the side of an animal's face, focusing on the deformable inwardly curved seal; FIG. 17B shows a close up of cross sectional view of FIG. 17A;

FIG. 18 shows a number of possible arrangements of the deformable lip portion of the mask body wall.

### DETAILED DESCRIPTION OF THE DRAWINGS

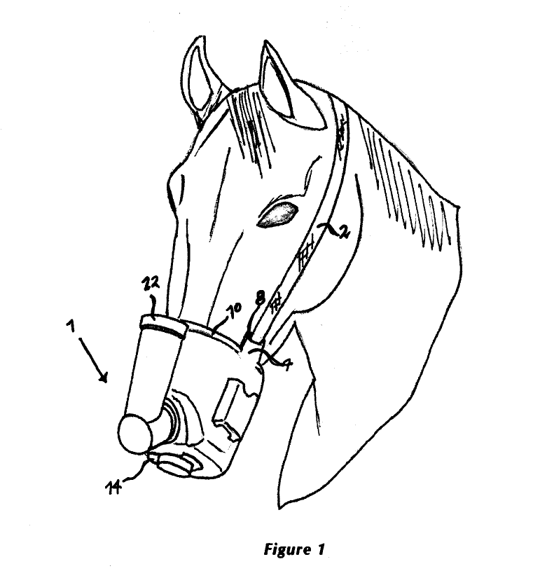

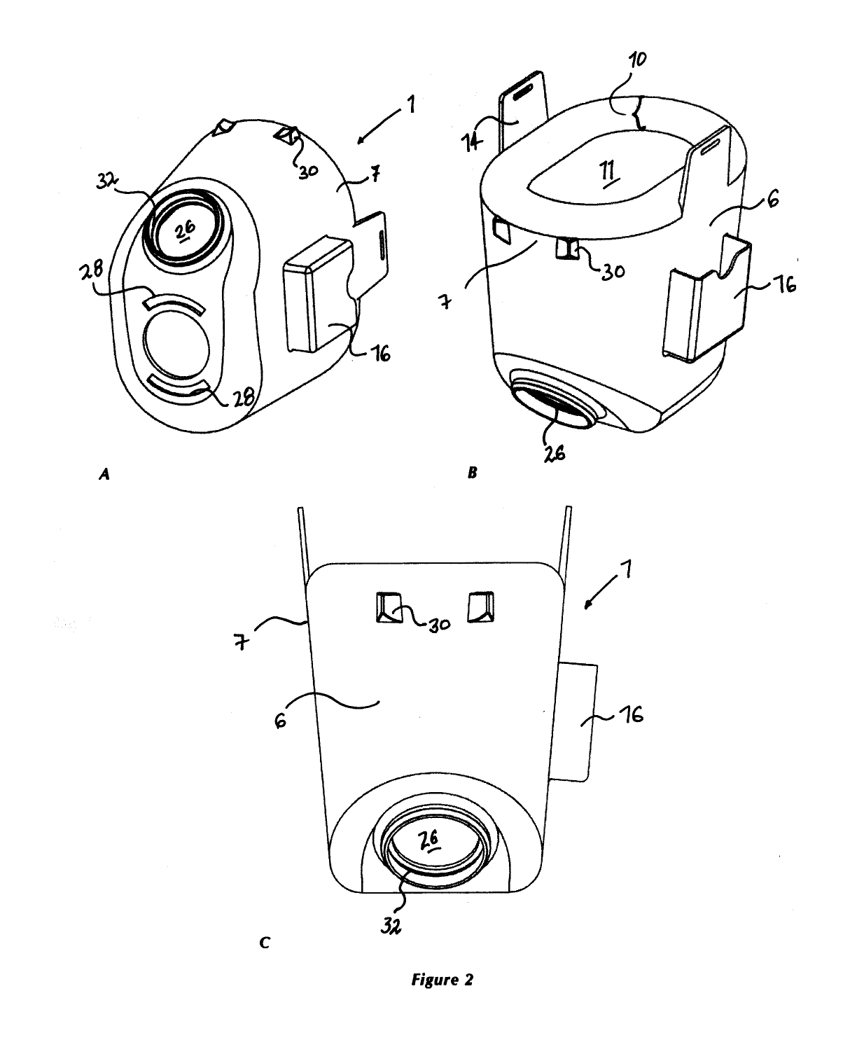

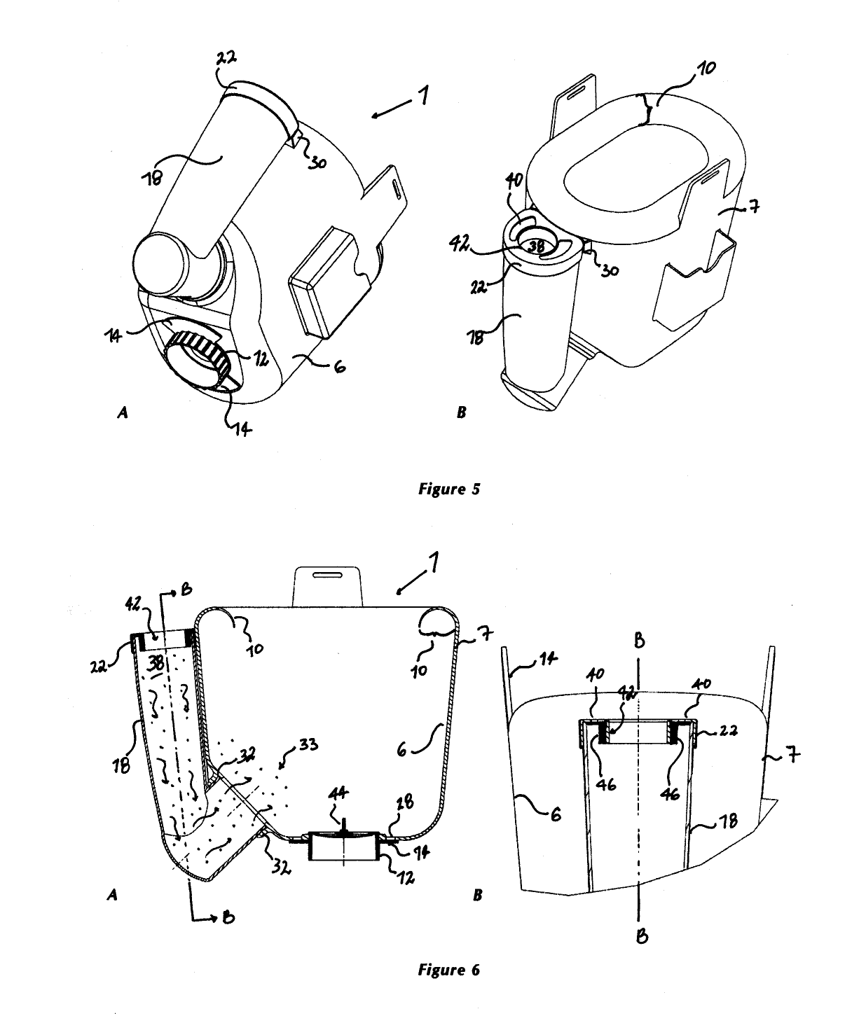

Referring now to the drawings and specifically FIGS. 1 to 18 inclusive and initially FIG. 1. FIG. 1 shows a specific example of an animal inhalation mask of the invention, illustrated generally by reference number 1 fitted over a horse's snout. In this example, the inhalation mask 1 is held in position on the horse's snout by adjustable headstrap 2. The headstrap 2 fits around the horse's head and sits behind its ears to hold the mask 1 in position. The headstrap 2 is attached to the mask 1 in this example by way of a tab 4 formed in the flexible mask housing 6. The tab 4 comprises an eyelet 8 through which the headstrap 2 may be secured. The skilled person will appreciate there is a corresponding tab 4′ on the opposite side of the inhalation mask 1. The unitary flexible housing 6 of the unitary mask 1 is evident from the illustration. The upper portion of the flexible housing 6 terminates in an inwardly turned and deformable lip 10 which functions to seal the mask 1 onto the animal's snout. The inwardly deformed lip 10 runs along the entire periphery of the opening 11 (shown in FIG. 2B) in the mask which is in turn formed by a continuation of the flexible mask housing 6 wall 7 (sidewall 7). In this specific example, the lower portion of the mask 1 has rotatable bypass valve port insert 12 installed. A pair of sealing flanges 14 which form part of the rotatable bypass valve port insert 12 are showing covering bypass port slots 28 which are located in the flexible mask housing 6. The flexible mask housing 6 also has a pocket 16 attached to the mask housing 6 for holding an electronic controller device 60 (shown in FIGS. 15A and 15B). FIG. 1 also shows an installed aerosol chamber 18 which is rotatably mounted onto an outlet 26 (shown in FIGS. 2A, 2B and 2C) provided in mask 1 through a snap-fit connection 20. The aerosol chamber inlet 42 (shown in FIG. 5A and FIGS. 6A and 6B) is covered by an aerosol chamber cap 22 which comprises valved inlets (not shown) and a port (not shown) adapted to accommodate a nebuliser head/aerosol generating device or alternative adaptors/accessories.

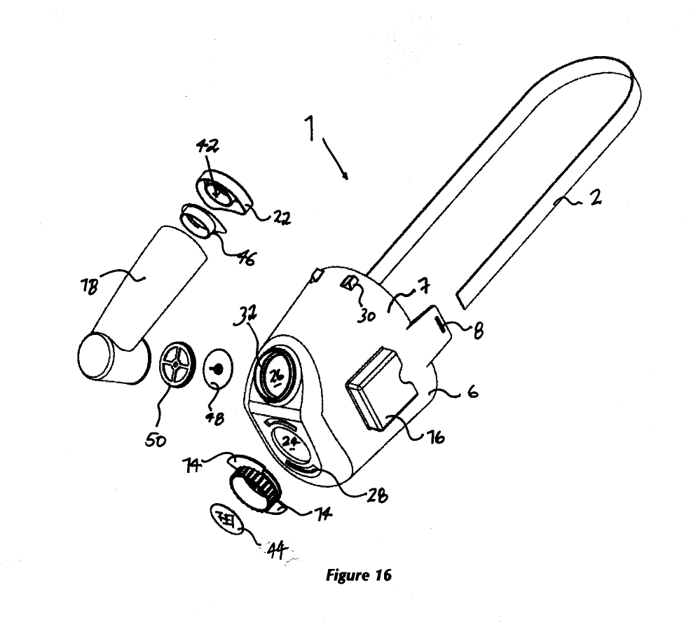

FIGS. 2A to 2C illustrate three different orientations of the flexible mask housing 6. Both mask outlet 24 and inlet 26 are clearly identifiable in FIG. 2A. The bypass port slots 28 are also observable in position about the mask outlet. FIG. 2B illustrates the inwardly turned deformable lip 10 running about the entire periphery of the flexible mask body 6. The inwardly turned deformable lip 10 provides the seal with the animal's snout when the mask 1 is positioned correctly and ready for use. All three figures show a pair of positioning stops 30 positioned towards the front of the mask body 6. These stops 30 assist in maintaining the initial upright position of the rotatable aerosol chamber 18 when it is installed onto the mask inlet 26 and on the horse's snout by providing a degree of resistance to rotation. The pair of stops 30 is shown positioned at the front of the mask body 6 and are located on the upper portion of the mask body near the inwardly turned deformable lip 10. A pair of headstrap tabs 4 for accommodating headstrap 2 extend upwards out of the upper portion of the mask body 6 and extend away from the inwardly turned deformable lip 10. Pocket 16 for accommodating an electronic controller 60 (shown in FIG. 13) are clearly shown on the left hand side of the mask 1 as it is viewed from the front on direction. Mask inlet 26 comprises a snap fit connector 32 arrangement which allow an aerosol chamber 18 to be removeably rotatably coupled to the inlet 26 for rotation thereon.

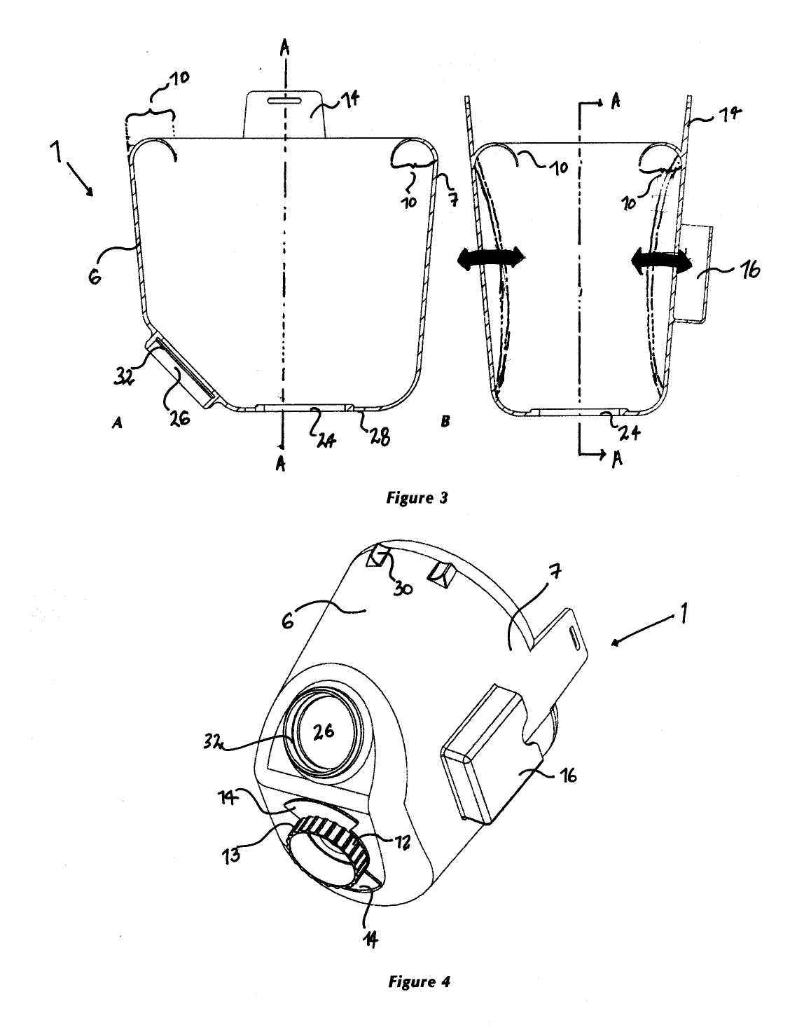

FIGS. 3A and B each illustrate sections through the mask 1. FIG. 3A is a section view along line A-A of FIG. 3B and identifies the inwardly turned deformable lip 10 running about the entire periphery of the flexible mask body 6. The mask inlet 26 and snap fit connector 32 is shown in this figure, as is the combined bypass port (24 & 28), which includes the outlet 24 and bypass port slots 28. FIG. 3B is a section view along line A-A of FIG. 3A. FIG. 3B additionally shows the pocket 16 on an external side of the left hand sidewall 7 of the mask housing 6 as it is viewed from the front end on, which is used to hold an electronic controller 60 (shown in FIG. 13) for a nebuliser/aerosol generating device.

FIG. 4 shows an example of the inhalation mask 1 of the invention having all of the features/components described thus far additionally with the rotatable bypass valve port insert 12 installed in the mask outlet 24. The rotatable bypass valve port insert 12 has gripping portions 13 around the circumference of the insert's outer walls and a pair of sealing flanges 14 which form an integral part of the rotatable bypass valve port insert 12. In the example shown, the rotatable bypass valve port insert 12 is in the closed position in which flanges 14 cover the bypass port slots 28 (see FIG. 2A). A valve 44 (shown in FIG. 6A) is installed in the centre of the rotatable bypass valve port insert 12.

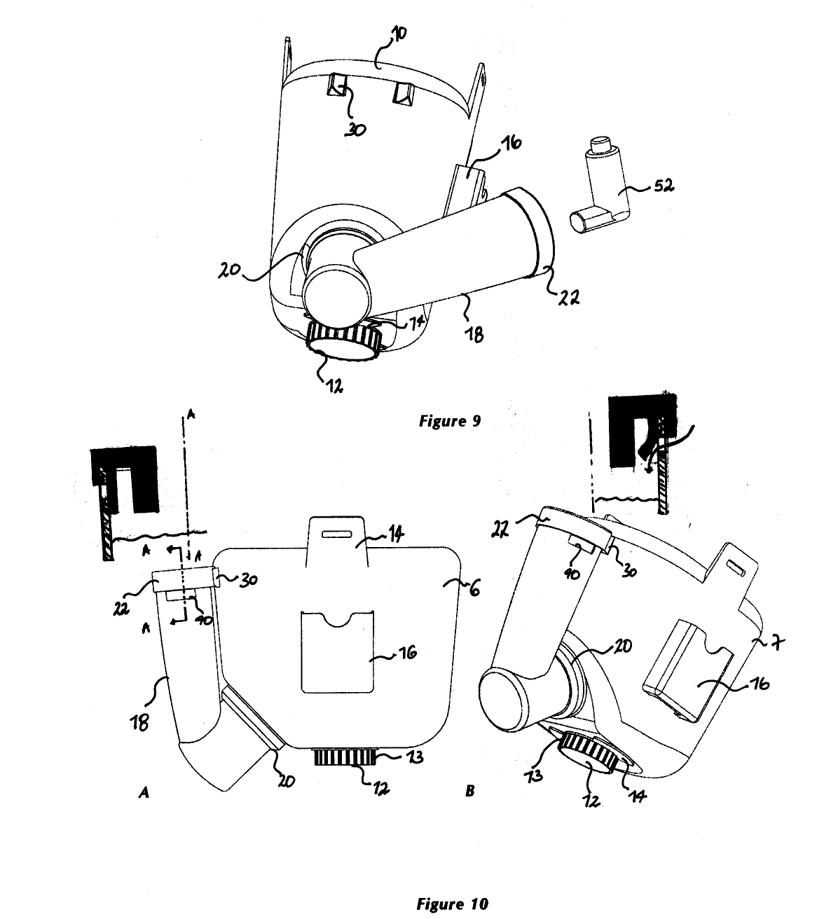

FIGS. 5A and 5B show two orientations of the mask 1 of the invention having the rotatable bypass valve port insert 12 installed in outlet 24 in the closed position and aerosol chamber 18 installed in the mask inlet 26. Stops 30 maintain the aerosol chamber 30 in the initial vertical position by resisting rotation movement. The operator/veterinarian may move the aerosol chamber to a substantially horizontal position by applying sufficient rotation pressure to overcome the stops 30. FIGS. 5A and 5B show aerosol chamber cap 22 in position on the aerosol chamber inlet 38. The aerosol chamber cap 22 fits removeably on top of the aerosol chamber inlet 38 and comprises a pair of air inlets 40 which are valved, and a port 42, adapted to accommodate a nebuliser head 58 (shown in FIG. 13) or other aerosol generating device such as an MDI 52 (shown in FIG. 9). FIGS. 6A and 6B each illustrate sectional views through the example of the mask 1 of the invention shown in FIG. 5A and FIG. 5B having an aerosol chamber 18 installed and a rotatable bypass valve port insert 12 and flanges 14 for covering bypass port slots 28 of the mask. Valve 44 installed in the rotatable bypass valve port insert 12 in the position illustrated in the figure. FIG. 6A is a section view through line B-B of FIG. 6B. The snap fit connector 32 that holds the aerosol chamber 18 in position is shown. The arrows indicate the flow of aerosol 33 into the mask 1 through aerosol chamber inlet 38 and mask inlet 26 respectively. FIG. 6B is a section view though line B-B of FIG. 6A but focusing on the aerosol chamber cap 22 and valve 46 which is in the closed position against air inlet valves 40. Port 42 is also clearly visible.

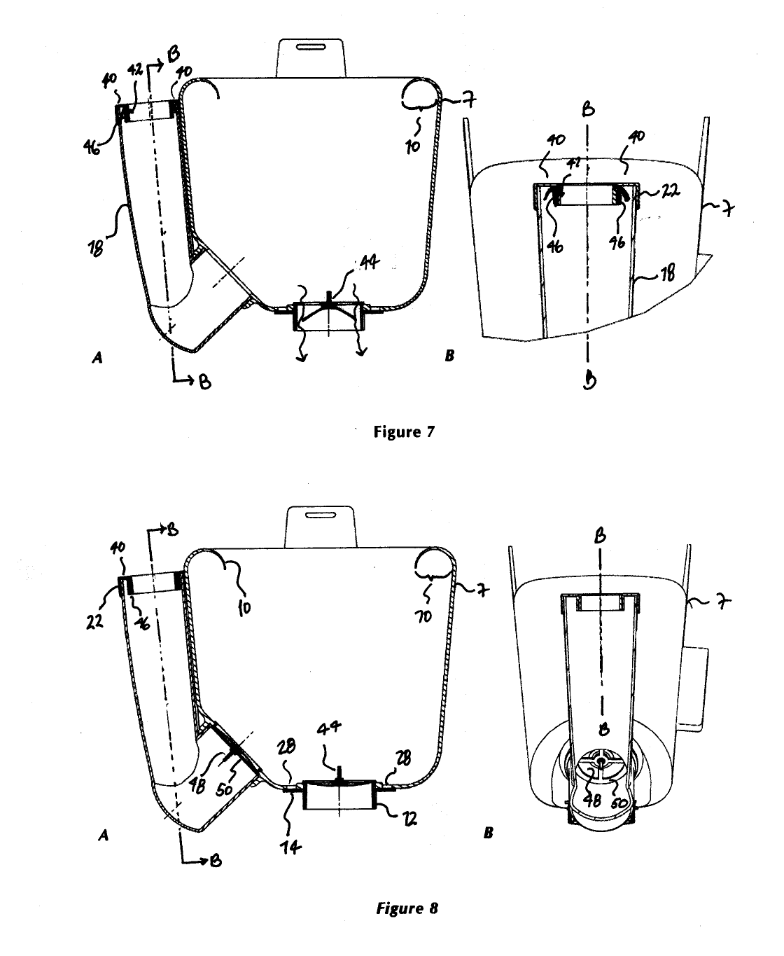

FIGS. 7A and B are respectively the same as FIGS. 6A and 6B but showing the valves 44 and 46 in the open position.

FIGS. 8A and 8B show optional mask inlet valve 48 in valve insert 50 installed in mask inlet 26. FIG. 8B shows a section view of the front of the mask along section line B-B. Optional mask inlet valve 48, valve insert 50 are clearly shown in the sectional view of FIG. 8B.

FIG. 9 shows the mask 1 of the invention with the aerosol chamber 18 after rotation out of stops 30 to a substantially horizontal position to the right hand side of the mask 1. The substantially horizontal position facilitates uses of an aerosol generating device such as MDI 52. MDI 52 fits into position within port 42 (not shown) of the aerosol chamber cap 22.

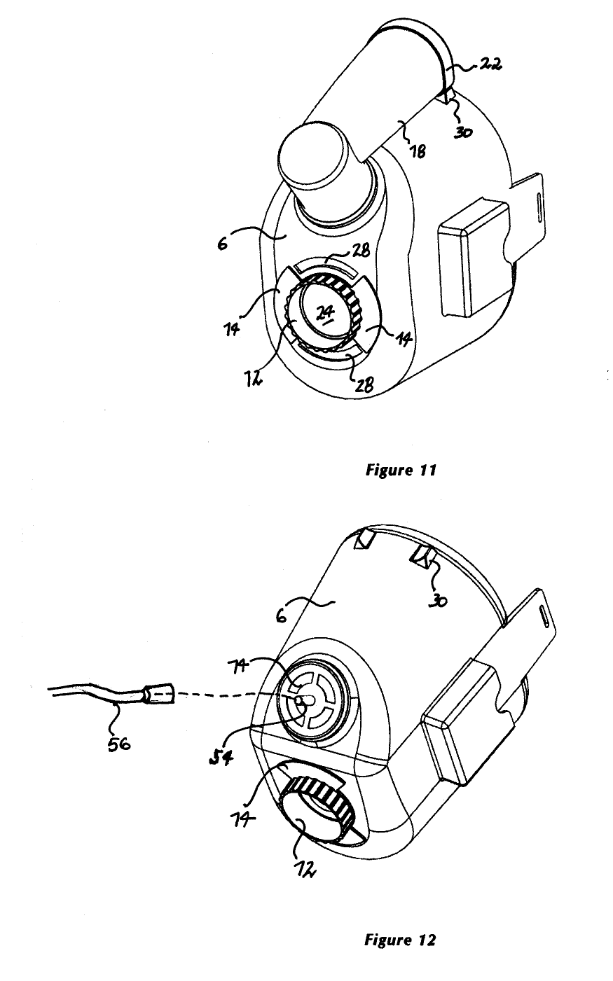

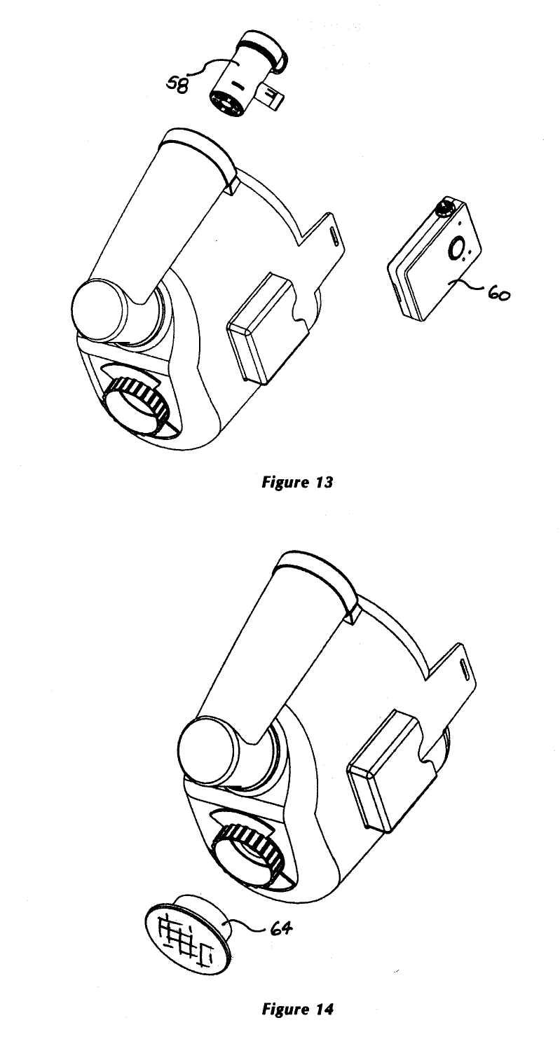

FIG. 10A shows a side view of mask 1 with aerosol chamber 18 in the vertical position and illustrates an alternative position for air inlets 40 in the aerosol chamber 18 side walls. FIG. 10B is equivalent to FIG. 5A but illustrates the alternative position for aerosol inlets 40 in the aerosol chamber 18 side walls.

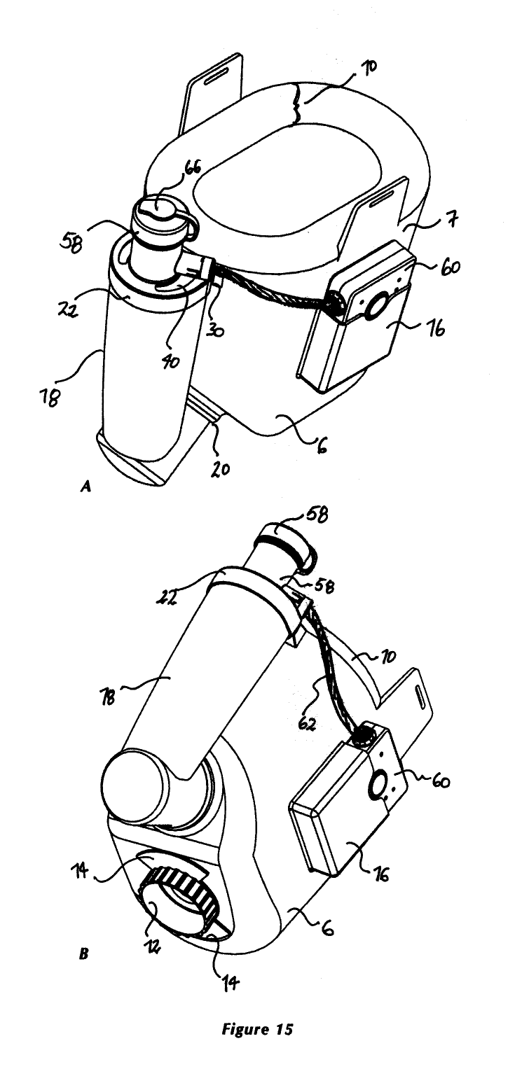

FIG. 11 is equivalent to FIG. 5A but shows the rotatable bypass valve port insert 12 installed in outlet 24 in the open position. Bypass port slots 28 are unhindered in this view and the flanges 14 of the rotatable bypass valve port insert 12 are rotated to the side to unobstruct the bypass port slots 28.

FIG. 12 shows a view of the mask 1 without aerosol chamber but with a gas (e.g. oxygen) port adaptor 54 installed into mask inlet 26 (not shown). A gas hose 56 connects the gas port adaptor 54 to a remote gas supply (not shown).

FIG. 13 show a mask of the invention as described with a nebuliser head 58 and electronic controller 60 for the nebuliser head 58 to be installed for use. An electronic communication cable connects the devices (not shown).

FIG. 14 shows an optional plug-in filter 64 which is adapted to be mountable onto the rotatable bypass valve port insert 12 if hazardous exhaust are likely.

FIGS. 15A and B show a mask of the invention as described with a nebuliser head 58 and electronic controller 60 for the nebuliser head 58. An electronic communication cable 62 connects the devices. The aerosol chamber air inlets 40 are clearly shown in FIG. 15A.

FIG. 16 shows an exploded view of the previously described components of mask 1 of the invention.

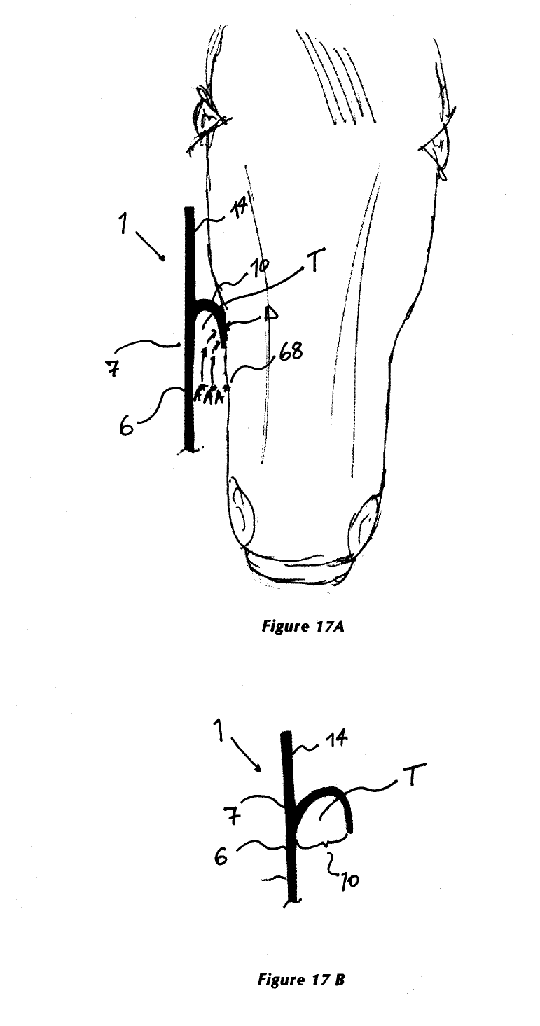

FIG. 17A shows a cross sectional view of part of the mask 1, along the side of the animal's face 68 and indicates the deformation of a portion D of the inwardly deformable lip 10 of the mask body 6 side wall 7 that occurs to form the air trap T, when the mask 1 fitted on the animal's snout. The arrows A* indicate the direction of backpressure which may be applied to the air trap T which enhances the seal; FIG. 17B shows the mask body 6 side wall 7, the inwardly curved portion of the deformable lip 10 curving away from the linear portion of the mask body 6 side wall 7.

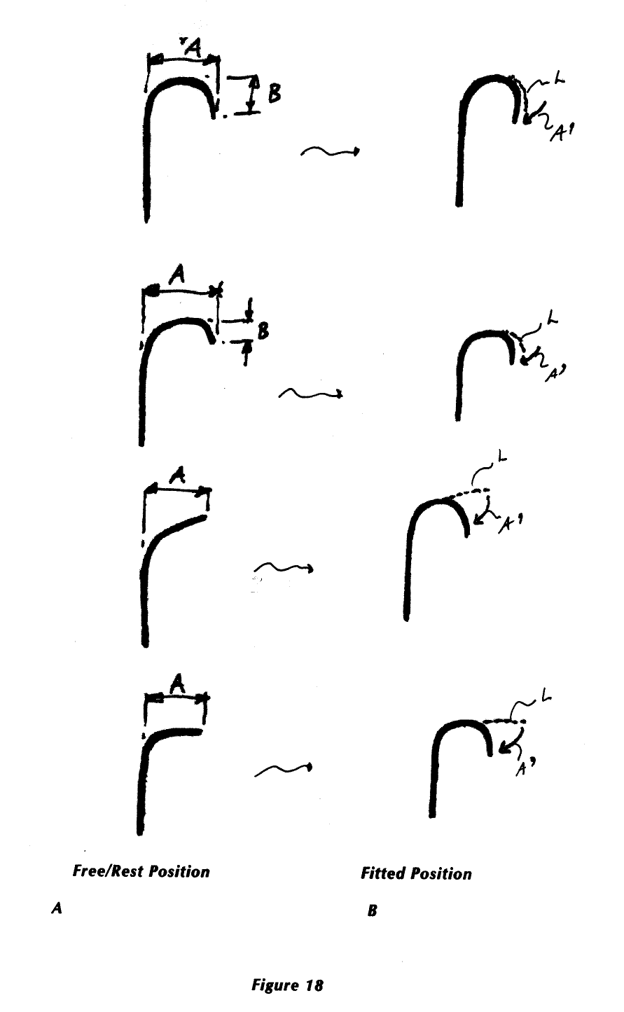

FIG. 18 shows a number of possible arrangements of the deformable lip 10 portion of the mask body 6 side wall 7. FIG. 18A shows the deformable lip in a number of possible rest positions (before the mask is fitted). As can be seen from the figure, various degrees of inwardly turned curvature can form the lip, e.g., (i) where the inwardly curved deformable lip has a substantially inturned portion B which is greater than or equal to approximately 50% of the length of portion A; (ii) shows a lip where the inwardly curved deformable lip has a substantially inturned portion B which less than or equal to approximately 50% of the length of portion A; (iii) shows a lip where the inwardly curved deformable lip does not have a curved inward portion B in the at rest state; (iv) shows a lip where the inwardly curved deformable lip having a portion B, which is curved inwards at an angle to be substantially perpendicular to side wall 7; The length A is the distance of the deviation away from the substantially linear mask body 6 wall 7. Length B is the length of the downward curve of the deformable mask wall 7. FIG. 18B shows corresponding deformed positions of the deformable lip when the mask is fitted in position. The greater the inward curvature of the lip when the mask is in the rest position, the less displacement from this position occurs when the mask is fitted. The broken lines L indicate the initial position of the lip when the mask is in the rest state, and the arrows A′ indicate the displacement from the normal rest position to the position when the mask is fitted in place.

In operation, the inhalation mask 1 of the invention can be prepared for use by assembling the necessary mask components. For example, if the aerosol chamber 18 is required, it can be prepared by fitting aerosol chamber cap 22 with fitted inlet valve 46 to the aerosol chamber inlet 38.

If required, the aerosol chamber 18 may be fitted with an optional aerosol outlet valve 48 if the chamber design so allows prior to attaching the aerosol chamber 18 to the mask inlet 26 through the corresponding snap fit connectors 32 on both chamber 18 and mask inlet 26. When the aerosol chamber 18 has been connected correctly, it should be rotatable on the mask housing body 6 within the inlet 26. The aerosol chamber can then be rotated to a substantially vertical position on the mask where it can be held in position by the pair of retaining stops 30.

The rotatable bypass valve port insert 12 and outlet valve 44 may be installed into the outlet space located in the mask body 6. If a hazardous medicament is to be administered, then the aerosol filter component 64 may be attached to the rotatable bypass valve port insert 12 to provide protection to the veterinarian or handler or nearby animals.

The adjustable headstrap 2 may then be added to the mask by passing its ends through eyelet 8 on tab 4 which is part of the flexible mask housing 6. When passed through the eyelet 8, the ends of the headstrap 2 can be secured together to attach it to the mask 1, on both sides.

In the case of a nervous animal, or one on which the mask 1 is to be used for the first time, bypass valve port insert 12 can be activated to the bypass position by rotation such that sealing flanges 14 do not obstruct the bypass slots 28 located towards the bottom of the mask housing body 6 before the mask in placed on the animal's snout. Activating the bypass port in this manner allows unrestricted breathing and may be less upsetting for the animal until it becomes accustomed to the inhalation mask 1.

The assembled mask 1 can then be carefully placed on the animal's snout ensuring the mouth and nostrils of the animal are well covered. When the animal has settled and appears comfortable with the mask 1, and it is in the correct position, the headstrap 2 can be further adjusted to ensure a comfortable and secure fit on the horse's head.

If a nebuliser head 58 is to be used to generate the aerosol/mist, the medicament can be placed inside the medicament cup and cup sealed by closing lid 66. The filled nebuliser head 58 may then be fitted into port 42 located on the aerosol chamber cap 22. Typically, the nebuliser head 58 is controlled by an electronic controller 60 which communicates with the nebuliser head 58 through connecting communication cable 62. The controller 60 may be inserted into the correspondingly sized pocket 16 on the mask housing body 6.

If an MDI 52 is to be used as the source of aerosol, a small amount of rotation pressure may be applied to the aerosol chamber 18 to rotate it within the mask inlet 26 such that it may be displaced from its vertical position in which it is maintained by securing stops 30. The aerosol chamber may then be rotated to a substantially horizontal position which will allow an MDI 52 to be used as mist generator.

When the veterinary/operator is ready to commence administration, rotatable bypass valve port insert 12 can be rotated to deactivate the bypass valve by moving the sealing flanges 14 over bypass slots 28 located towards the bottom of the mask housing. Deactivating the bypass port in this manner forces the animal to inhale air drawn in from the one way aerosol chamber inlets 40 and to exhale through the one way outlet valve 44 installed in the rotatable bypass valve port insert 12. If the mask is the correct size, and has been fitted on the animal's snout in the correct position, the inwardly deformed lip 10 which runs around the periphery of the open end of the mask housing 6 should positively engage with the animal's face to form a seal about the mask body 6 and face. The flexible nature of the mask 1 will indicate if a good seal has been formed, since the mask body 6 will pulsate or perceptibly rise and fall according to the horses breathing cycle. Generally, after the bypass port valve 48 has activated, when the horse exhales back pressure created improves the seal. When a good seal is indicated, the aerosol may be generated by the preferred means (nebuliser head 58 or the MDI 52). The generated aerosol then enters the aerosol chamber 18.

When the horse inhales, the one way outlet valve 44 remains closed and the one way inlet valves 46 and 48 (if installed) open and air is drawn into the aerosol chamber 18 where it mixes with the aerosol. As the horse inhales further, the aerosol air mixture is drawn into the interior of the mask body 6. Since the mask is sealed to the horse's snout, the aerosol/air mixture is completely drawn into the horse's lungs.

When the horse exhales, the one way inlet valves 46 and 48 (if installed) closes and the one way outlet valve 44 opens to allow expired air and aerosol to be driven out of the mask (through the aerosol filter 64, if installed). As the next respiratory cycle commences, the one way outlet valve 44 once again closes and the one way inlet valves 46 and 48 (if installed) opens to allow fresh air to be is drawn into the aerosol chamber 18. The cycle can be allowed to continue until the required dose has been administered to the horse. When treatment has been completed, the rotatable bypass valve port insert 12 can be rotated to deactivate the valves if desired and create an unrestricted breathing path for the animal. The inhalation mask 1 can then be removed after the adjustable headstrap is untied. The component parts can then be easily disassembled to facilitate inhalation mask 1 cleaning/disinfection.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

It will be readily apparent to one of ordinary skill in the art that the examples disclosed herein below represent generalised examples only, and that other arrangements and methods capable of reproducing the invention are possible and are embraced by the present invention.

1. An inhalation mask for fitting to a snout of an equine animal or an animal having an equine shaped snout and comprising:
a unitary mask body defining a housing with an open end which is adapted to overfit and accommodate the snout of the animal so that, when fitted to the animal, a nose, mouth and anterior portion of the animal's snout are entirely within the housing, the open end of the housing being an integral part of the housing and forming a deformable seal about the open end for sealing about the snout of the animal,
wherein the open end of the housing comprises a deformable lip which forms the deformable seal, the lip being formed by a continuation of a side wall forming the mask body, which when the mask is fitted, turns back inward upon itself, into the open end of the mask, so that the lip engages with the animal's snout to support the mask body on the snout.

2. A mask according to claim 1 wherein the deformable lip forms an air trap between the inwardly turned wall surface of the deformable lip and the inner wall of the mask body side wall.

3. A mask according to claim 1 wherein the mask body (6) is constructed of a material having sufficient flexibility to perceptibly rise and fall with respiration of an animal.

4. A mask according to claim 1 further comprising at least one inlet for allowing air into the mask during inhalation by the animal and at least one exhaust outlet for allowing venting during exhalation by the animal.

5. A mask according to claim 1 further comprising an aerosol chamber which is external to the mask and is in communication with the housing of the mask.

6. A mask according to claim 5 wherein the aerosol chamber is moveable relative to the mask so that the relative position of the chamber to the mask can be changed while the chamber remains in communication with the housing of the mask.

7. A mask according to claim 5 wherein at least one inlet for allowing air into the mask during inhalation by the animal is provided on the aerosol chamber.

8. An animal inhalation mask according to claim 4 wherein the inlet comprises at least one of an aerosol/air inlet valve, an aerosol holding chamber, a dust filter, an oxygen inlet adaptor, a flow sensor or monitoring sensor.

9. An animal inhalation mask according to claim 4 wherein the outlet comprises at least one of an exhaust outlet valve, a dust filter, a flow sensor, an exhaust aerosol filter or an exhaust gas measuring device.

10. An animal inhalation mask according to claim 9 wherein the exhaust outlet valve is located in the rotatable insert mountable in the housing exhaust outlet.

11. An animal inhalation mask according to claim 1 further comprising a bypass port in the housing.

12. An animal inhalation mask according to claim 11 wherein the bypass port further comprises an adjustable bidirectional valve.

13. An animal inhalation mask according to claim 12 wherein the adjustable bidirectional valve comprises at least one slot located in the housing and corresponding adjustable covering flange.

14. An animal inhalation mask according to claim 10, further comprising a bypass port in the housing, wherein the bypass port further comprises an adjustable bidirectional valve which comprises at least one slot located in the housing and the corresponding adjustable covering flange wherein the adjustable covering flange is part of the rotatable insert.

15. An animal inhalation mask according to claim 4 wherein the exhaust outlet and bypass port are the same.

16. An animal inhalation mask according to claim 1 for use in prevention of feeding or biting.

17. An inhalation mask for fitting to a snout of an equine animal or an animal having an equine shaped snout and comprising:
a unitary mask body defining a housing with an open end which is adapted to overfit and accommodate the snout of the animal so that, when fitted to the animal, a nose, mouth and anterior portion of the animal's snout are entirely within the housing, the open end of the housing being an integral part of the housing and forming a deformable seal about the open end for sealing about the snout of the animal,
wherein the open end of the housing comprises a deformable lip which forms the deformable seal, the lip being formed by a continuation of a side wall forming the mask body, which when the mask is fitted, turns back inward upon itself, into the open end of the mask, so that the lip engages with the animal's snout to support the mask body on the snout such that the mask is substantially self-supported on the animal's snout by an interference fit between the deformable lip and the animal's snout.

18. (canceled)
